(19) United States
(12) Patent Application Publication
(10) Pub. No.: US 2019/0008548 A1
(43) Pub. Date: Jan. 10, 2019

(54) ULTRASOUND MEDICAL DEVICE

(71) Applicant: OLYMPUS CORPORATION, Tokyo (JP)

(72) Inventors: Hiroshi ITO, Tokyo (JP); Shinji YASUNAGA, Tokyo (JP)

(73) Assignee: OLYMPUS CORPORATION, Tokyo (JP)

(21) Appl. No.: 16/130,365

(22) Filed: Sep. 13, 2018

Related U.S. Application Data
(63) Continuation of application No. PCT/JP2016/059629, filed on Mar. 25, 2016.

(51) Int. Cl. A61B 17/32; H01L 41/047; H01L 41/083; A61B 18/14

(52) U.S. Cl. CPC A61B 17/320092; H01L 41/0471; A61B 2018/00607; A61B 18/1442; H01L 41/083

(57) ABSTRACT

An ultrasound medical device includes: a treatment portion configured to treat a living tissue; and a piezoelectric transducer unit provided in the treatment portion and formed by laminating a plurality of piezoelectric elements, which is configured to excite ultrasound vibration, and a plurality of insulating layers and bonding each of the piezoelectric elements and the insulating layers through bonding layers. A thickness of each of the piezoelectric elements, the insulating layers, and a plurality of the bonding layers is set such that nodes of the ultrasound vibration are located other than in the bonding layers.

### CROSS REFERENCE TO RELATED APPLICATION

This application is a continuation application of PCT/JP2016/059629 filed on Mar. 25, 2016, the entire contents of which are incorporated herein by this reference.

### BACKGROUND OF INVENTION

### 1. Field of the Invention

The present invention relates to an ultrasound medical device that is provided with an ultrasound vibration device and is used for endoscopic surgery, and that grasps a living tissue to perform coagulation/dissection or the like on the living tissue.

### 2. Description of the Related Art

A surgical treatment device, for example as disclosed in Japanese Patent Application Laid-Open Publication No. 2003-135479, has been hitherto known as one of treatment instruments to be used in endoscopic surgery.

This conventional surgical treatment device is provided with a probe for transmitting ultrasound vibration generated in an ultrasound transducer to a distal end, and grasping means, which makes a pair with the distal end of the probe, including a press member that is openable/closable to grasp the living tissue.

The conventional surgical treatment device includes a heater, which is a heat generation element for causing a distal end of the probe to generate heat, in the grasping means and is configured so as to be able to coagulate and dissect the grasped living tissue due to friction heat generated by ultrasound vibration and heat applied by the heater.

More specifically, the surgical treatment device of Japanese Patent Application Laid-Open Publication No. 2003-135479 includes: a jaw, which makes a pair with a distal end of an ultrasound treatment probe and is openable/closable to grasp a living tissue; and a heater as a heat generation pattern at the distal end of the ultrasound treatment probe. The heater provided at the distal end of the ultrasound treatment probe is energized to generate heat so that the grasped living tissue can be coagulated and dissected.

### SUMMARY OF THE INVENTION

An ultrasound medical device in one aspect of the present invention includes: a treatment portion configured to treat a living tissue; and a piezoelectric transducer unit provided in the treatment portion and formed by laminating a plurality of piezoelectric elements, which is configured to excite ultrasound vibration, and a plurality of insulating layers and bonding each of the piezoelectric elements and the insulating layers through bonding layers. A thickness of each of the piezoelectric elements, the insulating layers, and a plurality of the bonding layers is set such that nodes of the ultrasound vibration are located other than in the bonding layers.

### BRIEF DESCRIPTION OF THE DRAWINGS

FIG. 1 is a perspective view showing a whole configuration of an ultrasound medical device in one aspect of the present invention;

FIG. 2 is a perspective view showing a configuration of a grasping portion of the ultrasound medical device in the one aspect of the present invention;

FIG. 3 is a sectional view showing a configuration of an ultrasound probe of the grasping portion in the one aspect of the present invention;

FIG. 4 is a perspective view showing a configuration of each of the piezoelectric elements bonded with a blade in the one aspect of the present invention;

FIG. 5 is a sectional view of the grasping portion in a state of treating a living tissue in the one aspect of the present invention;

FIG. 6 is a view for explaining thicknesses of respective members and positions of nodes of vibration at the time of using high-order resonance of a whole laminate made up of a piezoelectric transducer unit and a blade, in the one aspect of the present invention;

FIG. 7 is a view for explaining thicknesses of respective members and positions of nodes of vibration at the time of using high-order resonance of a rectangular piezoelectric body of a first modification in the one aspect of the present invention; and

FIG. 8 is a view for explaining thicknesses of respective members and positions of nodes of vibration at the time of using first-order resonance of the whole laminate made up of the piezoelectric transducer unit and the blade, in the one aspect of the present invention.

### DETAILED DESCRIPTION OF THE PREFERRED EMBODIMENTS

Hereinafter, the present invention will be described using the drawings.

In the following description, the drawings based on each embodiment are schematic, and it should be noted that a relationship between a thickness and a width of each part, a ratio of thicknesses of respective parts, and the like are different from actual ones. Dimensional relations and ratios in some parts included in in each drawing may also be different among the drawings.

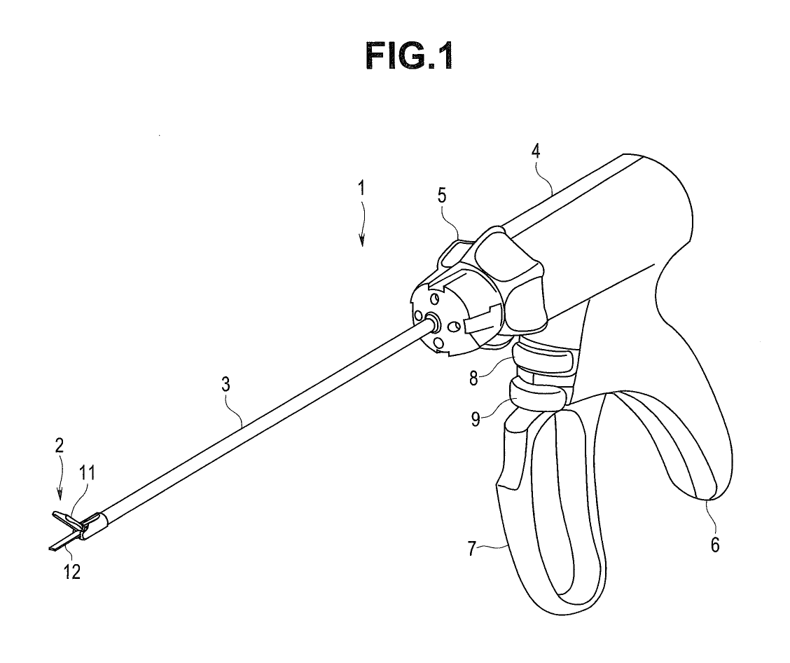

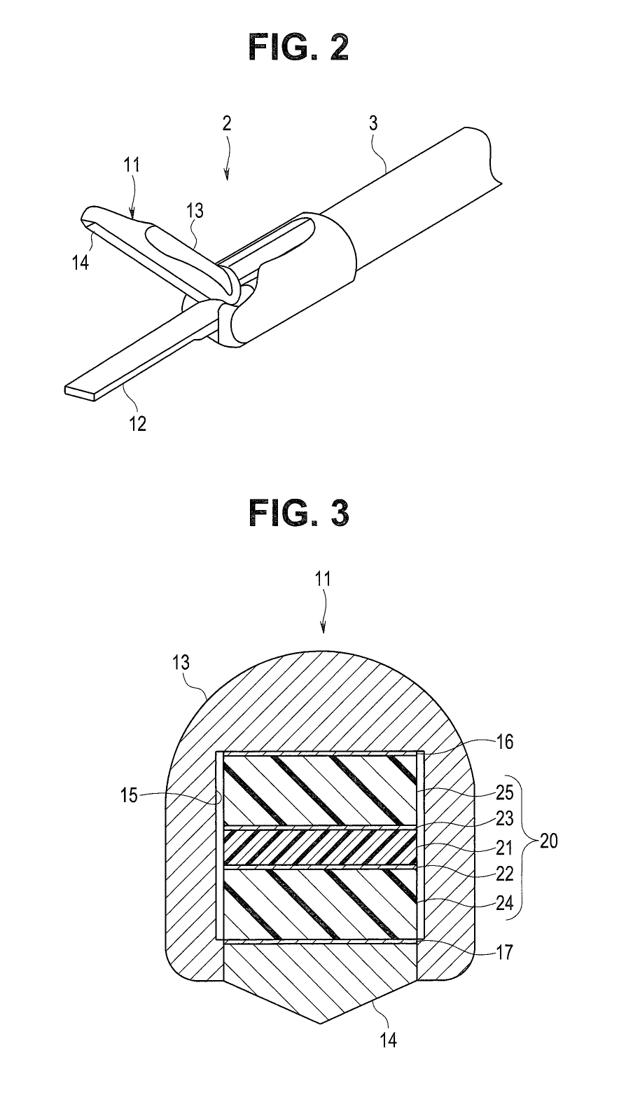

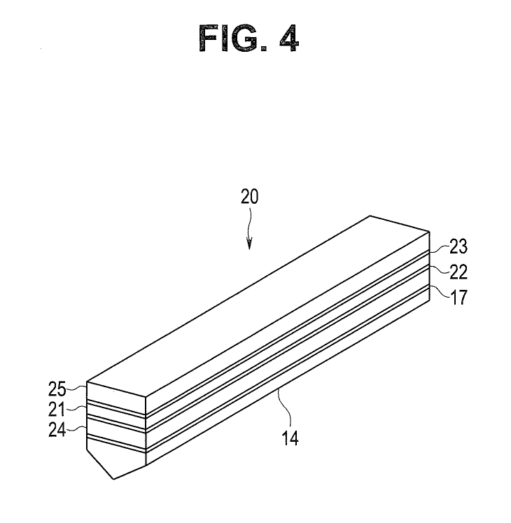

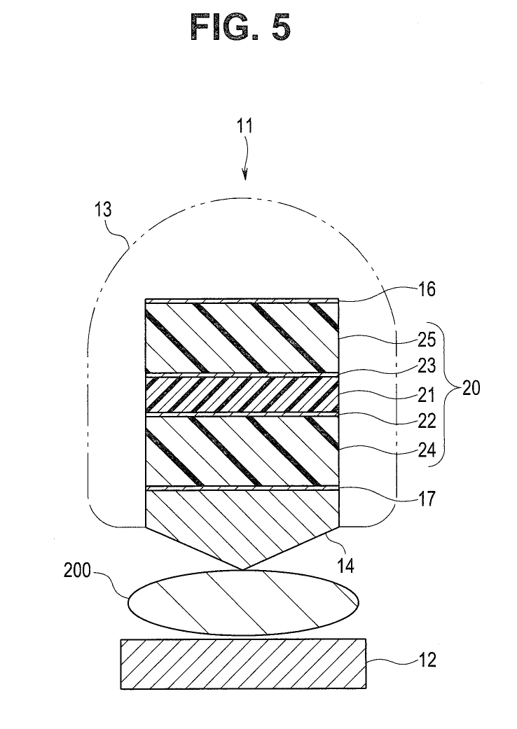

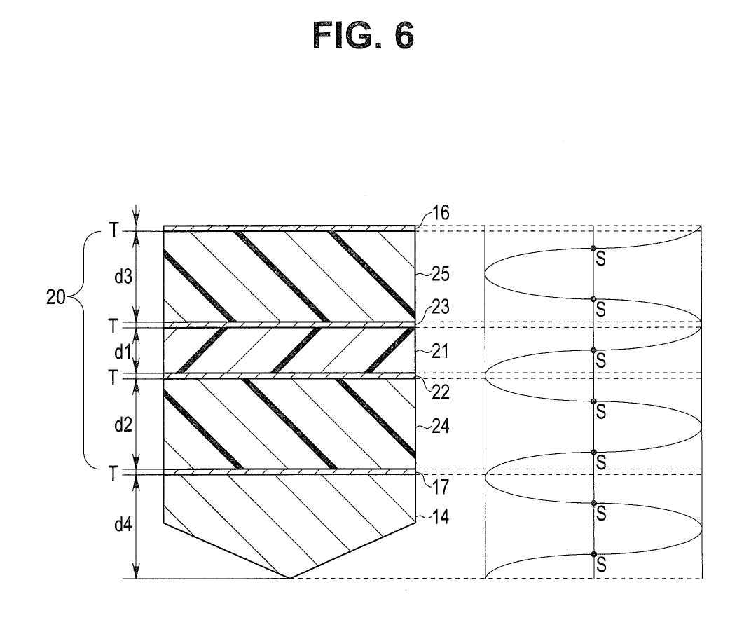

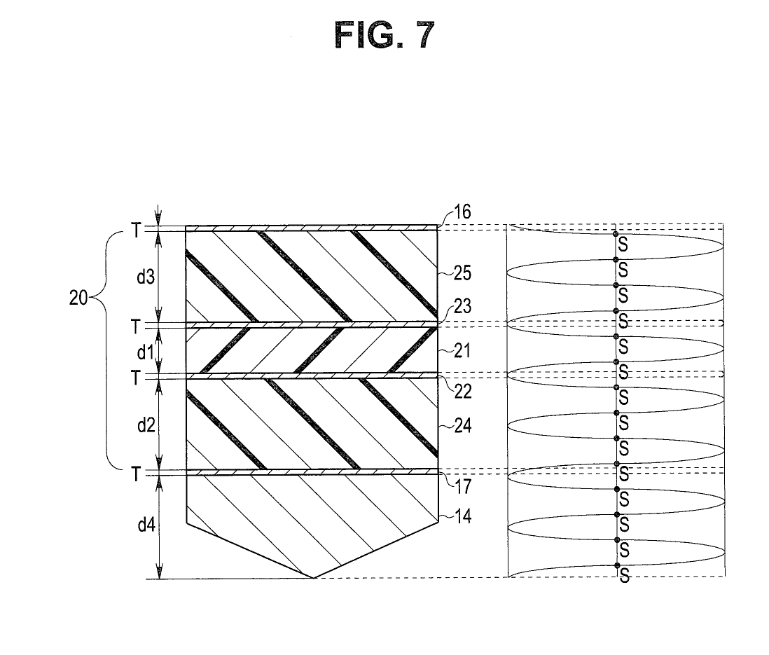

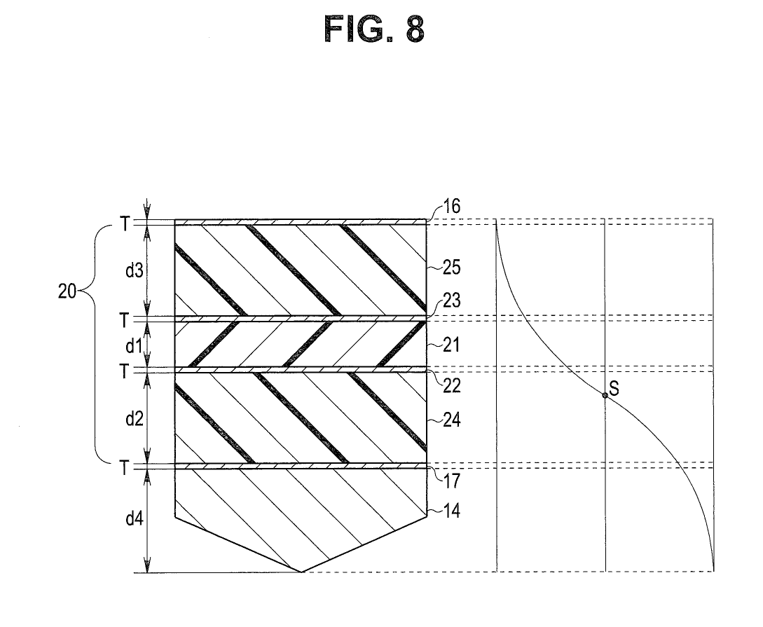

FIG. 1 is a perspective view showing a whole configuration of an ultrasound medical device in one aspect of the present invention. FIG. 2 is a perspective view showing a configuration of a grasping portion of the ultrasound medical device. FIG. 3 is a sectional view showing a configuration of an ultrasound probe of the grasping portion. FIG. 4 is a perspective view showing a configuration of each of the piezoelectric elements bonded with a blade. FIG. 5 is a sectional view of the grasping portion in a state of treating a living tissue. FIG. 6 is a view for explaining thicknesses of respective members and positions of nodes of vibration at the time of using high-order resonance of a whole laminate made up of a piezoelectric transducer unit and a blade. FIG. 7 is a view for explaining thicknesses of respective members and positions of nodes of vibration at the time of using high-order resonance of a rectangular piezoelectric body of a first modification. FIG. 8 is a view for explaining thicknesses of respective members and positions of nodes of vibration at the time of using first-order resonance of the whole laminate made up of the piezoelectric transducer unit and the blade.

### (Ultrasound Medical Device)

First, a configuration of an ultrasound medical device in the present embodiment will be described below.

As shown in FIG. 1, an ultrasound medical device 1 being an ultrasound treatment instrument as a device for surgery is configured including a grasping portion 2, an insertion tube portion 3, and an operation portion, sequentially from a distal end, the grasping portion 2 being a distal end portion of a probe that grasps a living tissue to perform coagulation (stanching)/dissection, bonding, anastomosis, or the like on the living tissue 4 as a treatment portion.

The grasping portion 2 includes an ultrasound probe 11 as an ultrasound blade, and a plate-like jaw 12 formed of metal having biocompatibililty, or the like. Note that the ultrasound probe 11 is openably/closably provided with respect to the jaw 12.

With the ultrasound probe 11 operating with respect to the jaw 12 as thus described, while being obliquely in contact with a living tissue disposed on the jaw 12, the ultrasound probe 11 extends a contact region.

Therefore, a region on which ultrasound acts on the living tissue can be changed, so that it is possible to restrict the action region in accordance with situations, and further to reduce damage caused by a periphery of the treatment portion on the living tissue.

The insertion tube portion 3 provided with this grasping portion 2 is made of a rigid metal pipe, and a rotational operation member 5 connected and fixed to the proximal end portion is pivotally operated from a neutral position to be freely pivotable around a longitudinal axis, so as to horizontally swing in a range of about a half rotation (180°).

Hence the grasping portion 2 is rotated around the longitudinal axis of the insertion tube portion 3 in conjunction with the pivoting of the insertion tube portion 3. That is, in the ultrasound medical device 1 here, the pivoting operation of the rotational operation member 5 enables the grasping portion 2 to swing and pivot in such a direction as to easily grasp the living tissue.

The operation portion 4 is configured including: the rotational operation member 5 that rotationally operates the insertion tube portion 3 described above; a fixed handle 6 to be held during use; a movable handle 7 that performs an operation to open and close the ultrasound probe 11; a first handle switch 8 with which ultrasound and high-frequency output are operated for performing coagulation/dissection, bonding, anastomosis, or the like on the living tissue such as blood vessel; and a second handle switch 9 with which a high-frequency output is operated for coagulating the living tissue or stanching blood. Note that a cable for power supply, not shown here, is connected to the operation portion 4.

Alternatively, the ultrasound medical device 1 may be configured such that a bending portion is provided in a rear part of the grasping portion 2 of the insertion tube portion 3, and an operation member that performs an operation to bend the bending portion is provided in the operation portion 4.

As described above, the ultrasound medical device 1 of the present embodiment constitutes ultrasound coagulation/dissection forceps being an ultrasound treatment instrument that grasps the living tissue with the grasping portion 2 to perform coagulation (stanching)/dissection, bonding, anastomosis, or the like on the living tissue.

### (Ultrasound Probe)

Next, a configuration of the ultrasound probe 11 in the present embodiment will be described below.

As shown in FIG. 2, the ultrasound probe 11 includes: an exterior block 13 being a metal block made of metal having biocompatibility; a blade 14 that is exposed from a lower end of the exterior block 13 and has a pentagonal shape in cross section here, and a piezoelectric transducer unit 20 shown in FIG. 3 is built in the exterior block 13.

As shown in FIG. 3, the blade 14 is disposed in contact with the piezoelectric transducer unit 20 via the bonding layer 17 so as to enable transmission of vibration from the piezoelectric transducer unit 20, functions as a transmission portion that transmits ultrasound generated from the piezoelectric transducer unit 20, and is formed of a metal block made up of a titanium alloy, such as TAB6400 (ASTM B348 Gr5), having biocompatibililty.

Further, in the present embodiment, the example has been shown where the blade 14 is formed in the pentagonal shape in cross section, but the blade 14 may only be configured so as to be able to transmit vibration from the piezoelectric transducer unit to the living tissue as a target.

When dissection performance is to be improved, it is desirable in terms of efficiency that a polygon, which includes a part having an acute angle in cross section, be in contact with the living tissue.

Therefore, in the present embodiment, the blade 14 is provided such that an acute angle side in cross section faces the jaw 12. Further, when bonding performance and anastomosis performance are to be emphasized, it is preferable that a surface of the blade 14 which comes into contact with the living tissue be a flat surface, namely a sectional surface shape of the blade 14 be a rectangular shape.

As shown in FIG. 3, the piezoelectric transducer unit 20 provided inside the ultrasound probe 11 is disposed as bonded to a concave groove portion 15 formed in the exterior block 13 via a bonding layer 16, an upper surface of which becomes a bonding portion.

The groove portion 15 of the exterior block 13 is in contact only with an upper surface of the piezoelectric transducer unit 20 via the bonding layer 16, and for electrical insulation from the piezoelectric transducer unit 20, the groove portion 15 is spaced from the piezoelectric transducer unit 20 by a predetermined distance so as not to come into contact with a crosswise side surface of the piezoelectric transducer unit 20 and a longitudinal surface, though not shown here, of the piezoelectric transducer unit 20. The piezoelectric transducer unit 20 is then bonded to the blade 14 via the bonding layer 17, a lower surface of which becomes a bonding portion.

Note that a space between the groove portion 15 of the exterior block 13 and the piezoelectric transducer unit 20 may be filled with highly heat-resistant, epoxy resin having heat resistance of 300° C., or a ceramic inorganic filler. Alternatively, an air layer can be provided between the exterior block 13 and the piezoelectric transducer unit 20 in such a configuration where the bonding layer 16 is eliminated and the exterior block 13 is bonded only to a side surface of the blade 14.

As thus described, inside the exterior block 13, the piezoelectric transducer unit 20 of the present embodiment is provided with an upper-side portion surrounded by the exterior block 13 and a lower surface covered with the blade 14.

That is, in the ultrasound probe 11, the exterior block 13 and the blade 14 constitute a metal mass member for the piezoelectric transducer unit 20. Especially, the blade 14 has a function to transmit ultrasound generated by the piezoelectric transducer unit 20.

### (Piezoelectric Transducer Unit)

Next, the piezoelectric transducer unit 20 as an ultrasound transducer will be described below.

As shown in FIG. 4, the piezoelectric transducer unit 20 includes: a rectangular piezoelectric body 21 in a shape of a surface rectangular plate; bonding layers 22, 23 laminated so as to sandwich the rectangular piezoelectric body 21; and insulating layers 24, 25 being two insulating plates laminated so as to further sandwich the bonding layers 22, 23.

Note that the whole of the piezoelectric transducer unit 20 here is fainted into a substantially square columnar shape by laminating the rectangular piezoelectric body 21, each of the bonding layers 22, 23, and each of the insulating layers 24, 25 such that four corners and four sides of each of the laminated are aligned. That is, each of the rectangular piezoelectric body 21, the bonding layers 22, 23, and the insulating layers 24, 25 has a rectangular shape with substantially the same front and rear surfaces.

When coagulation (stanching)/dissection, bonding, anastomosis, or the like is to be performed on a living tissue by using heat, the living tissue needs to be heated to a temperature of 200 to 300° C. For performing the above treatment with each of piezoelectric elements provided at a distal end of the treatment instrument, each of the piezoelectric elements needs to be not deteriorate even at 200 to 300° C., and a piezoelectric material having a Curie point of 600° C. or higher is required.

As measures for the above, it is considered that a transducer for generating ultrasound vibration and a heater being a heat generation element (heat generation pattern) for heating the living tissue are separately provided, but forming such a configuration causes a problem of high pricing on a product, and hence it is naturally preferable that the number of members for generating energy be one.

Therefore, for the rectangular piezoelectric body 21 as the piezoelectric material, lithium niobate single crystal (LiNbO₃) of piezoelectric single crystal is used which has a Curie point of about 1200° C., and piezoelectric characteristics of which hardly deteriorate while in use over time at the time of bonding.

In a crystal orientation called 36-degree Y-cut of lithium niobate single crystal (LiNbO₃), an electromechanical coupling factor kt of the transducer in a thickness direction is a value comparable to an electromechanical coupling factor kt of lead zirconate titanate (PZT) which has hitherto been used for the piezoelectric material, so that an electric signal can be efficiently converted into ultrasound vibration.

The value of the electromechanical coupling factor kt changes depending on the crystal orientation and becomes maximal at a certain angle. The specific crystal orientation of 36-degree Y-cut has been cited here in consideration of availability in the market, but the crystal orientation is not restricted to the above, and a crystal orientation close to 36-degree Y-cut may be used in such a range that the electromechanical coupling factor does not decrease greatly.

Further, lithium niobate single crystal (LiNbO₃) is one of non-lead piezoelectric materials having a high mechanical Q factor suitable for an ultrasound transducer for high output and is environmentally excellent due to no use of lead.

Note that the piezoelectric single crystal material used for the rectangular piezoelectric body 21 is not restricted to lithium niobate single crystal (LiNbO₃), but may be a piezoelectric single crystal material such as lithium tantalate (LiTaO₃).

The bonding layers 22, 23 bond the insulating layers 24, 25 to upper and lower surfaces of the rectangular piezoelectric body 21, and one of the bonding layers 22, 23 is a positive electrode layer while the other is a negative electrode layer. Wires, not shown, for supplying drive signals to the piezoelectric transducer unit 20 are electrically connected to the bonding layers 22, 23.

Bonding each of the rectangular piezoelectric body 21, the insulating layers 24, 25, the exterior block 13, and the blade 14 requires heat resistance and durability for constituting the ultrasound probe 11 as a device used in the ultrasound medical device 1 that performs treatment using ultrasound.

Hence each of the bonding layers 16, 17, 22, 23 for bonding each member desirably performs bonding by brazing and soldering with conductivity so that the ultrasound probe 11 maintains strong bonding force even at temperatures rising during operation, such as 200 to 300° C., to prevent attenuation of ultrasound excited (generated) by the piezoelectric transducer unit 20 and that especially the bonding layers 22, 23 also serve as the electrode layers. A brazing member, such as gold-tin (AnZn) or gold-germanium (AuGe), having a high melting point and conductivity is suitable.

Note that the insulating layers 24, 25 are provided for electrically insulating the exterior block 13 and the blade 14 from the drive signal that drives the piezoelectric transducer unit 20 and are preferably made of a material absorbing little ultrasound and having large strength, and zirconium dioxide (ZrO₂) is suitable, for example.

As thus described, in the piezoelectric transducer unit 20, the insulating layer 24, the rectangular piezoelectric body 21, and the insulating layer 25 are sequentially laminated via the bonding layers 22, 23 so as to insulate the exterior block 13 and the blade 14 from the drive signal that flows in one of the bonding layers 22, 23 and returns to the other of the bonding layers 22, 23.

As shown in FIG. 5, in the ultrasound medical device 1 of the present embodiment configured as above, a living tissue 200 such as blood vessel can be pinched on the jaw 12 by using the ultrasound probe 11, and the piezoelectric transducer unit 20 is driven so that coagulation (stanching)/dissection, bonding, anastomosis, or the like can be performed on the living tissue 200 by vibration and heat.

### (Action)

In the ultrasound medical device 1 of the present embodiment, ultrasound at a frequency of several tens of MHz is generated in the piezoelectric transducer unit 20, and the ultrasound generated in the piezoelectric transducer unit 20 is transmitted to the living tissue 200 via the blade 14.

The living tissue 200 absorbs the ultrasound from the blade 14, a temperature of the living tissue 200 thus increases, and coagulation/dissection, bonding, anastomosis, or the like is performed.

At this time, there is an advantage that the more ultrasound the living tissue 200 absorbs, the lower the power supply to the piezoelectric transducer unit 20, which is necessary for the treatment, can be made.

On the other hand, there is a disadvantage that, when the drive frequency becomes higher, more ultrasound is absorbed by the living tissue 200, to cause reduction in distance at which the ultrasound from the blade 14 can be propagated.

There is a trade-off relation between the above advantage and disadvantage, and the ultrasound medical device 1 is required to propagate the ultrasound to the living tissue 200 at approximately several millimeters so as to pinch and treat the living tissue 200 with the grasping portion 2.

Therefore, in order to propagate the ultrasound at such a distance that the living tissue 200 can be treated and to increase absorption of the ultrasound into the living tissue 200, the frequency of the ultrasound is appropriately from 10 to 50 MHz.

Then, for propagating stable ultrasound to the living tissue 200, it is desirable to use high-order resonance of a whole laminate configured of the piezoelectric transducer unit 20 and the blade 14.

There is an advantage that by using the high-order resonance of the whole laminate configured of the piezoelectric transducer unit 20 and the blade 14, stable resonance characteristics can be obtained and the ultrasound drive can be performed easily.

However, at the time of using the high-order resonance of the laminate configured of the piezoelectric transducer unit 20 and the blade 14, as shown in FIG. 6, a plurality of nodes S of vibration are generated in the laminate.

Vibration stress becomes maximal at each of the places of the plurality of nodes S, and hence the nodes S of vibration are set to be located within a thickness of the rectangular piezoelectric body 21, the insulating layers 24, 25, and the blade 14 such that the nodes S of vibration are not located in the bonding layers 16, 17, 22, 23 which are bonding portions bonding different types of materials.

More specifically, a thickness d1 of the rectangular piezoelectric body 21, thicknesses d2, d3 of the insulating layers 24, 25, a thickness d4 of the blade 14, and a thickness T of each of the bonding layers 16, 17, 22, 23 are set to be predetermined thicknesses such that the nodes S of vibration are located within the thickness of the rectangular piezoelectric body 21, the insulating layers 24, 25 and the blade 14.

Ideally, it is desirable to set each of the thicknesses d1 to d4 and T such that antinodes of the vibration are located in the respective bonding layers 16, 17, 22, 23.

As thus described, the ultrasound medical device 1 of the present embodiment is configured such that the rectangular piezoelectric body 21 being each of the piezoelectric elements is driven at a high-order resonance frequency of the whole laminate configured of the piezoelectric transducer unit 20 and the blade 14 in the exterior block 13 provided in the ultrasound probe 11 of the grasping portion 2, to generate ultrasound vibration of several tens of MHz.

In the ultrasound medical device 1, the thickness d1 of the rectangular piezoelectric body 21, the thicknesses d2, d3 of the insulating layers 24, 25, the thickness d4 of the blade 14, and the thickness T of each of the bonding layers 16, 17, 22, 23 are set such that each of the bonding layers 16, 17, 22, 23 bonding the different types of materials for the blade 14 and the piezoelectric transducer unit 20 is located between the nodes S of vibration.

In the ultrasound medical device 1 configured as above, heat generation inside the living tissue 200 can be used by efficiently heating the living tissue 200, grasped by the grasping portion 2 provided at a distal end of the insertion tube portion 3, through ultrasound absorption, and as compared with heating by the conventional heat generating element such as the heater, it is possible to more uniformly heat from the surface to the inside, and to quickly and simply perform treatment (coagulation (stanching)/dissection, bonding, anastomosis, or the like), and thereby to reduce invasion of heat to a surface of the living tissue 200.

Differently from the conventional device, in the ultrasound medical device 1, the piezoelectric transducer unit 20 that vibrates at ultrasound frequency is provided in the grasping portion 2 at the distal end of the insertion tube portion 3, and it is thus possible to improve flexibility in swing and shape of the grasping portion 2. Even with a configuration where a bending portion is provided in the insertion tube portion 3, it is similarly possible to improve flexibility in bending operation.

Further, the ultrasound medical device 1 is not provided with a heat generating element such as the heater other than the ultrasound transducer as in the conventional device, thus enabling reduction in product cost as compared with the conventional device.

Moreover, in the ultrasound medical device 1, the high-order resonance of the laminate configured of the piezoelectric transducer unit 20 and the blade 14 is used, and the plurality of nodes S of vibration at which the vibration stress in the laminate becomes maximal are located at places different from the respective bonding layers 16, 17, 22, 23 which bond the different types of materials.

Hence in the ultrasound medical device 1, it is possible to reduce damage on each of the bonding layers 16, 17, 22, 23 bonding the different types of materials which are caused by vibration stress and to improve durability performance against ultrasound vibration excited (generated) in the ultrasound medical device 1.

From the above description, the ultrasound medical device 1 of the present embodiment is configured such that appropriate coagulation (stanching)/dissection, bonding, anastomosis, or the like is quickly and simply performed in accordance with a treated region of the treatment portion of the living tissue 200 to reduce invasion to the living tissue and have a low price and durability.

### (First Modification)

As described above, in the piezoelectric transducer unit 20, the rectangular piezoelectric body 21 is used which is each of piezoelectric elements produced from a wafer with a crystal orientation called 36-degree Y-cut of lithium niobate single crystal (LiNbO₃) that has a very high Curie point and efficiently obtains vibration in a thickness direction.

In the above case, a frequency coefficient of the rectangular piezoelectric body 21 provided in the piezoelectric transducer unit 20 is 3.3 MHz·mm, and hence, when the drive frequency is assumed to be 40 MHz in use of first-order resonance of the rectangular piezoelectric body 21, the thickness d1 of the rectangular piezoelectric body 21 is 82.5 μm, which is very small.

As above, when the thickness d1 of the rectangular piezoelectric body 21 is small, the strength becomes small to require care in handling at the time of assembly, and a defect such as breaking of the rectangular piezoelectric body 21 may occur.

Therefore, in the ultrasound medical device 1 of the present modification, the thickness d1 of the rectangular piezoelectric body 21 is made larger by using high-order resonance of the rectangular piezoelectric body 21 being each of the piezoelectric elements, to cause the rectangular piezoelectric body 21 to have appropriate strength.

For example, by driving the rectangular piezoelectric body 21 at a drive frequency of 40 MHz in a fifth-order resonance mode, the thickness d1 of the rectangular piezoelectric body 21 can be made about 410 μm which is five times as large as in the case of the first-order resonance, so that it is possible to cause the rectangular piezoelectric body 21 to have appropriate strength.

Also at the time of using the high-order resonance of the rectangular piezoelectric body 21 as in the present modification, as shown in FIG. 7, a plurality of nodes S of vibration are generated in the laminate configured of the piezoelectric transducer unit 20 and the blade 14.

Therefore, also here, the nodes S of vibration are set to be located within the thickness of the rectangular piezoelectric body 21, the insulating layers 24, 25, and the blade 14 such that the nodes S of vibration are not located in the bonding layers 16, 17, 22, 23 which are bonding portions bonding the different types of materials, and ideally, the thickness d1 of the rectangular piezoelectric body 21, the thicknesses d2, d3 of the insulating layers 24, 25, the thickness d4 of the blade 14, and the thickness T of each of the bonding layers 16, 17, 22, 23 are set such that the antinodes of the vibration are located in the respective bonding layers 16, 17, 22, 23.

As a result, in addition to the action effect described above, the ultrasound medical device 1 need not make the thickness d1 of the rectangular piezoelectric body 21 excessively small, thus enabling improvement in yield in manufacturing of the piezoelectric transducer unit 20.

### (Second Modification)

As shown in FIG. 8, the first-order resonance of the laminate configured of the piezoelectric transducer unit 20 and the blade 14 may be used.

Also at the time of using the first-order resonance of the laminate configured of the piezoelectric transducer unit 20 and the blade 14, the thickness d1 of the rectangular piezoelectric body 21, the thicknesses d2, d3 of the insulating layers 24, 25, the thickness d4 of the blade 14, and the thickness T of each of the bonding layers 16, 17, 22, 23 are set such that the nodes S of the vibration are not located in the respective bonding layers 16, 17, 22, 23 which are bonding portions bonding the different types of materials.

Further, in the embodiment and each modification described above, the grasping portion 2 may have such a configuration where the piezoelectric transducer unit 20 and the blade 14 may also be provided on the jaw 12 side facing the ultrasound probe 11 to provide two piezoelectric transducer units 20, or a configuration where the piezoelectric transducer unit and the blade 14 are provided only on the jaw 12 side which is a fixed jaw.

In the above description, lithium niobate single crystal (LiNbO₃) forming the rectangular piezoelectric body 21 has been configured to use the crystal orientation called 36-degree Y-cut and vertically vibrate, but this configuration is not restrictive, and the crystal orientation (place orientation) may be changed to cause the rectangular piezoelectric body 21 to vibrate horizontally.

The invention described in the above embodiment is not restricted to the embodiment and the modifications, and various modifications can be made in a scope not deviating from the gist of the invention in an implementation phase. Further, the above embodiment includes various stages of inventions, and appropriate combination of a plurality of disclosed constituent features enables extraction of various inventions.

For example, when the described problem can be solved and the described effect can be obtained even if some constituent features are deleted from all the constituent features shown in the embodiment, the configuration from which some constituent features have been deleted can be extracted as the invention.

What is claimed is:

1. An ultrasound medical device, comprising:
a treatment portion configured to treat a living tissue; and
a piezoelectric transducer unit provided in the treatment portion and formed by laminating a plurality of piezoelectric elements configured to excite ultrasound vibration and a plurality of insulating layers, and bonding each of the piezoelectric elements and the insulating layers through bonding layers,
wherein a thickness of each of the piezoelectric elements, the insulating layers, and a plurality of the bonding layers is set such that nodes of the ultrasound vibration are located other than in the bonding layers.

2. The ultrasound medical device according to claim 1, wherein the thickness of each of the piezoelectric elements, the insulating layers, and the plurality of bonding layers is set such that antinodes of the ultrasound vibration are located in the plurality of bonding layers.

3. The ultrasound medical device according to claim 1, further comprising
a metal block bonded to the piezoelectric transducer unit via a bonding portion and configured to propagate the ultrasound vibration to the living tissue,
wherein the nodes of the ultrasound vibration are located other than in the bonding portion interposed between the metal block and the piezoelectric transducer unit.

4. The ultrasound medical device according to claim 2, further comprising
a metal block bonded to the piezoelectric transducer unit via a bonding portion and configured to propagate the ultrasound vibration to the living tissue,
wherein the nodes of the ultrasound vibration are located other than in the bonding portion interposed between the metal block and the piezoelectric transducer unit.

5. The ultrasound medical device according to claim 1, wherein the thickness of each of the piezoelectric elements is such a thickness that a drive frequency of the piezoelectric transducer unit is a high-order resonance frequency of each of the piezoelectric elements.

6. The ultrasound medical device according to claim 2, wherein the thickness of each of the piezoelectric elements is such a thickness that a drive frequency of the piezoelectric transducer unit is a high-order resonance frequency of each of the piezoelectric elements.

7. The ultrasound medical device according to claim 3, wherein each of the piezoelectric elements is driven at a predetermined frequency at which a whole laminate made up of the piezoelectric transducer unit and the metal block resonates in a high order.

8. The ultrasound medical device according to claim 4, wherein each of the piezoelectric elements is driven at a predetermined frequency at which a whole laminate made up of the piezoelectric transducer unit and the metal block resonates in a high order.

9. The ultrasound medical device according to claim 1, wherein each of the piezoelectric elements is formed of a piezoelectric single crystal.

10. The ultrasound medical device according to claim 2, wherein each of the piezoelectric elements is formed of a piezoelectric single crystal.
